(19) United States
(12) Patent Application Publication
(10) Pub. No.: US 2025/0001035 A1
(43) Pub. Date: Jan. 2, 2025

(54) DISPENSER FOR A VOLATILE LIQUID COMPOSITION

(71) Applicant: The Procter & Gamble Company, Cincinnati, OH (US)

(72) Inventors: Ronald David TURNER, Walton, KY (US); Pei LI, Eschborn (DE); Faiz Feisal SHERMAN, Mason, OH (US)

(21) Appl. No.: 18/754,253

(22) Filed: Jun. 26, 2024

Related U.S. Application Data
(60) Provisional application No. 63/523,705, filed on Jun. 28, 2023.

(51) Int. Cl. A61L 9/12

(52) U.S. Cl. CPC A61L 9/127; A61L 9/122

(57) ABSTRACT

A dispenser for a volatile liquid composition includes a housing; a reservoir holding the liquid composition; a transport member for the liquid composition; and an evaporative member in fluid communication with the transport member. The evaporative member includes a wickable mesh. A method for dispensing a volatile liquid composition using a dispenser includes the step of wetting the evaporative member with the liquid composition; and evaporating the composition from the evaporative member.

### TECHNICAL FIELD

The present disclosure is in the field of dispensers for volatile liquid compositions. In particular, it relates to a dispenser comprising a transport member and an evaporative member.

### BACKGROUND

There are a variety of volatile composition dispensers available on the market today, including aerosol and pump sprayers, non-energized and energized dispensers that utilize a wick, diffusers, and the like, for delivering a volatile composition, such as a perfume composition or an insecticide, into the air. Energized dispensers, powered through a wall outlet (plug-in) tend to perform better than battery-powered dispensers. However, plug-in dispensers may have drawbacks. For example, the user does not have flexibility to place the dispenser anywhere in a room, it has to be placed close to a plug in the wall, this might not be the most appropriate place in terms of aesthetic or in terms of optimum position for the user to enjoy the dispensed composition.

Another potential limitation of prior art devices is that devices often comprise membranes that limit the diffusion of certain types of volatile materials.

There remains a need for improved devices that emit volatile compositions into the atmosphere.

### SUMMARY

According to a first aspect of the present disclosure, there is provided a dispenser for a volatile liquid composition, the dispenser comprises a housing; a reservoir containing a liquid volatile composition, a transport member in fluid communication with said liquid volatile composition; and an evaporative member in fluid communication, preferably in contact, with the transport member. The reservoir, the transport member and the evaporative member are preferably positioned within said housing. The evaporative member is situated downstream from the transport member, preferably in contact with the transport member. The evaporative member comprises a wickable mesh.

According to another example, there is provided a dispenser for a volatile liquid composition, the dispenser comprises a housing; a reservoir containing a liquid volatile composition, a transport member in fluid communication with said liquid volatile composition; and an evaporative member. The reservoir, the transport member and the evaporative member are preferably positioned within said housing. The dispenser provides certain evaporation rate using a relative low power or current input.

### BRIEF DESCRIPTION OF DRAWINGS

The above-mentioned and other features and advantages of the present disclosure, and the manner of attaining them, will become more apparent and the present disclosure itself will be better understood by reference to the following description of various examples of the present disclosure taken in conjunction with the accompanying drawings, wherein:

FIG. 1 is an exploded front perspective view of a dispenser in accordance with the examples of the present disclosure.

FIG. 2 is a graphical depiction of the wicking time of mesh samples in accordance with the examples of the present disclosure.

FIG. 3 is a graphical depiction of the average release rate of mesh samples in accordance with the examples of the present disclosure.

### DETAILED DESCRIPTION

Various examples will now be described to provide an overall understanding of the principles of the structure, function, manufacture, and use of the dispensers and methods disclosed herein. One or more examples of these examples are illustrated in the accompanying drawings. Those of ordinary skill in the art will understand that the devices and methods specifically described herein and illustrated in the accompanying drawings are non-limiting example examples and that the scope of the various examples of the present disclosure is defined solely by the claims. The features illustrated or described in connection with one example can be combined with the features of other example examples. Such modifications and variations are intended to be included within the scope of the present disclosure.

The dispenser can be used to dispense a volatile liquid composition, such as a fragrance, an insecticide or any other volatile liquid composition, including those that help with sleep, breathing, coughing, congestion, etc., to an area surrounding the dispenser, Preferably the volatile liquid composition is a fragrance. The dispenser comprises a housing, the housing comprises a reservoir holding a volatile liquid composition, a transport member and an evaporative member comprising a mesh. As its name suggests, the transport member transfers the volatile liquid composition from the reservoir to the evaporative member, from where the volatile liquid composition is distributed to the surrounding environment.

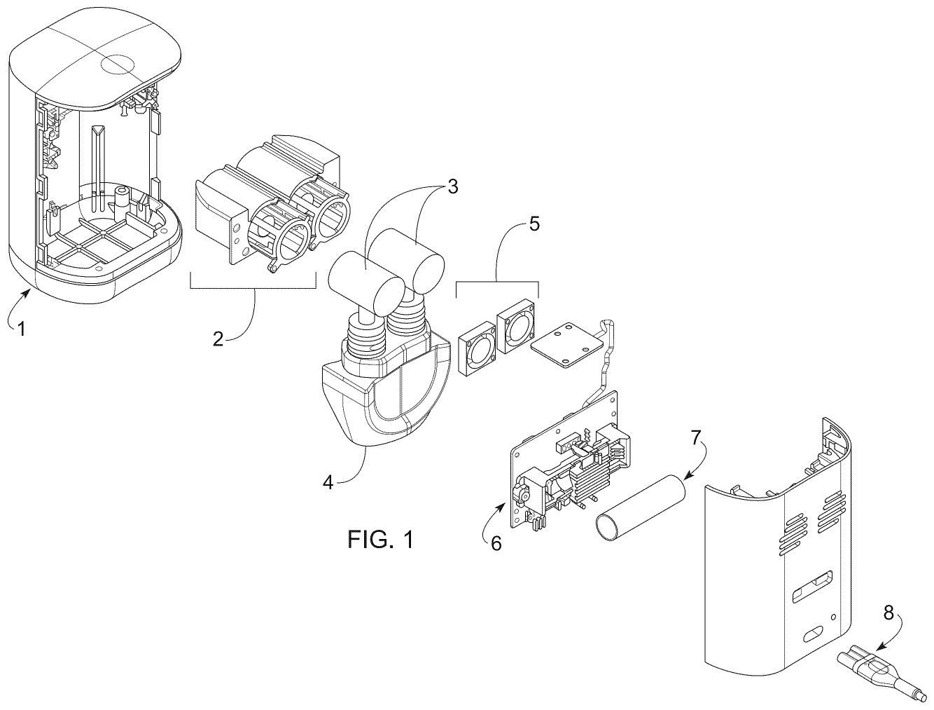

Referring to FIG. 1, a dispenser in accordance with the present disclosure comprises a housing 1 comprising a reservoir 4 a transport member under the evaporative member 3 comprising a mesh. The housing 1 can optionally comprise diffusion assistance means 5.

### Housing

The housing 1 may be configured to contain many or all of the elements of the dispenser. The housing 1 may be comprised of a single element or from multiple elements that are joined together to define an interior chamber. The dispenser showed in FIG. 1 has a dual chamber reservoir. The housing 1 may take any shape and may be composed of various materials, such as plastic, metal, resin, and the like.

The dispenser may comprise a housing 1 for housing the reservoir 4, the transport member and the evaporative member. The housing 1 may be formed from a single part or from multiple parts that are joined together to define at least one chamber. The device shown in FIG. 1 comprises a two-piece structure that is joined to form a first chamber enclosing all the elements of the dispenser. In some examples, the housing defines a single chamber. The chamber may comprise diffusion assistance means, such as a fan 5. The diffusion assistance means can be mounted anywhere in housing 1 with an air flow path that channels the airflow to maximize it through the evaporative member 3.

The housing is sized such that, when the diffusion assistance means, for example The air exchange rate is fully programmable to desired rate duty cycle for desired mg/hr volatile dosing rate.

The reservoir can be replenished once the volatile liquid has been evaporated.

The housing 1 can also comprise a movable door (not shown) that can be opened to remove and/or replace the reservoir. The housing 1 can also comprise a plug assembly 8 formed with the housing or in electrical communication with the housing, for example, by way of an electrical cord. 8 can also be thought of an inductive charger base to charge the dispenser. Alternative the housing might comprise a battery 7. 6 is the PCB control board that stores all needed memory for the operation of the fan 5, the charging of battery 7, driving any other means of energy such as electric current on the evaporative member 3 to rise the temperature or LED with different wavelengths, such as UV or IR, to heat the fluid composition on the evaporative member 3. The PCB 6 can also have sensors such as ambient light detection, human motion sensors such as radar, accelerometers, VOC sensors, wireless transmitters and wireless receivers (Bluetooth, WiFi, etc).

### Reservoir

The reservoir 4 may be configured to contain from about 5 milliliters (mL) to about 100 mL, from 5 to 50 mL, from 5 to 30 mL, from 5 to 20 mL, from 5 to 15 mL, from 5 to 10 mL, from 10 to 100 mL, from 10 to 50 mL, from 10 to 30 mL, from 10 to 20 mL, from 10 to 15 mL, from 15 to 100 mL, from 15 to 50 mL, from 15 to 30 mL, from 15 to 20 mL, from 20 to 100 mL, from 20 to 50 mL, from 20 to 30 mL, from 30 to 100 mL, from 30 to 50 mL, from 50 to 100 mL, and any values within the foregoing ranges or any ranges created thereby, of fluid composition. Preferably from about 10 mL to about 50 mL, more preferably from about 15 mL to about 30 mL of fluid composition. The dispenser may be configured to have multiple reservoirs, with each reservoir containing the same or different fluid composition and same or different fluid volumes. The reservoir can be made of any suitable material for containing a fluid composition including glass, plastic, metal, or the like. The reservoir may be transparent, translucent, or opaque or any combination thereof. For example, the reservoir may be opaque with a transparent indicator of the level of fluid composition in the reservoir.

### Transport Member

The transport member is in fluid communication with the reservoir 4 for delivering the volatile composition contained therein to the evaporative surface 3. The transport member may be in fluid communication with the base of the reservoir 4 in order to dispense substantially all of the volatile composition contained within the reservoir 4. The transport member may be defined by a first end portion, a second end portion, and a central portion separating the first and second end portions. The first end portion of the transport member may be disposed in the reservoir and may be in fluid communication with the volatile composition. At least a portion of the transport member is disposed within the interior of the reservoir. A portion of the transport member may extend to the exterior of the reservoir. The first end portion may contact the base of the reservoir. The transport member may be configured to direct the volatile composition from the reservoir 4 to the evaporative surface 3.

The transport member may be configured in various ways. For example, the transport member may be configured as a tube having an outer wall and a hollow interior. The tube may be in the form of a capillary tube. Preferably, the transport member comprises a wick, the wick may be defined by a length and a diameter or width, depending on the shape. The wick may have various lengths. For example, the length of the wick may be in the range of about 1 millimeter (“mm”) to about 100 mm, preferably from about 5 mm to about 75 mm, more preferably from about 10 mm to about 50 mm. In examples, the length of the wick may range from 1 to 100 mm, from 1 to 75 mm, from 1 to 50 mm, from 1 to 40 mm, from 1 to 30 mm, from 1 to 20 mm, from 1 to 10 mm, from 1 to 5 mm, from 5 to 100 mm, from 5 to 75 mm, from 5 to 50 mm, from 5 to 40 mm, from 5 to 30 mm, from 5 to 20 mm, from 5 to 10 mm, from 10 to 100 mm, from 10 to 75 mm, from 10 to 50 mm, from 10 to 40 mm, from 10 to 30 mm, from 10 to 20 mm, from 20 to 100 mm, from 20 to 75 mm, from 20 to 50 mm, from 20 to 40 mm, from 20 to 30 mm, from 30 to 100 mm, from 30 to 75 mm, from 30 to 50 mm, from 30 to 40 mm, from 40 to 100 mm, from 40 to 75 mm, from 40 to 50 mm, from 50 to 100 mm, from 50 to 75 mm, from 75 to 100 mm, and any values within the foregoing ranges or any ranges created thereby. The wick may have various diameters or widths. For example, diameter or width of the wick may be at least 1 mm, preferably at least 2 mm, more preferably at least 4 mm, most preferably least 6.5 mm. The diameter or width of the wick may range from 1 to 15 mm, from 1 to 10 mm, from 1 to 8 mm, from 1 to 7 mm, from 1 to 6.5 mm, from 1 to 5 mm, from 1 to 4 mm, from 1 to 2 mm, from 2 to 15 mm, from 2 to 10 mm, from 2 to 8 mm, from 2 to 7 mm, from 2 to 6.5 mm, from 2 to 5 mm, from 2 to 4 mm, from 4 to 15 mm, from 4 to 10 mm, from 4 to 8 mm, from 4 to 7 mm, from 4 to 6.5 mm, from 4 to 5 mm, from 5 to 15 mm, from 5 to 10 mm, from 5 to 8 mm, from 5 to 7 mm, from 5 to 6.5 mm, from 6.5 to 15 mm, from 6.5 to 10 mm, from 6.5 to 8 mm, from 6.5 to 7 mm, from 7 to 15 mm, from 7 to 10 mm, from 7 to 8 mm, from 8 to 15 mm, from 8 to 10 mm, from 10 to 15 mm, and any values within the foregoing ranges or any ranges created thereby.

The transport member may be composed of various materials, including plastic, glass, metal, rubber, silicone, and combinations thereof. The transport member may also be comprised of a porous or semi-porous wick that is wrapped in a non-air permeable outer wrap. The wick may be composed of various materials and methods of construction, including, but not limited to, bundled fibers which are compressed and/or formed into various shapes via overwrap (such as a non-woven sheet over-wrap) or made of sintered plastics such as PE, HDPE or other polyolefins. For example, the wick may be made from a plastic material such as polyethylene or a polyethylene blend.

### Evaporative Member

The volatile liquid composition evaporates from the evaporative member 3 into the air. The evaporative member comprises a mesh that is capable of wicking the liquid, once the liquid has wetted the evaporative member, the volatile liquid composition evaporates. The evaporative member is capable of wicking the liquid and yet prevents free flow of liquid out of the evaporative member. The evaporative member helps to control the evaporation rate of the liquid volatile composition. The evaporative member also helps the diffusion of the volatile liquid without requiring a lot of energy. This is a real advantage for dispensers that are not plug in to the mains and dispensers that operate with low power batteries.

Wicking is the spontaneous flow of a liquid in a porous substrate driven by capillary forces. The capillary force of the evaporative member should be greater or equal to the capillary force of the transport member.

The evaporative member comprises a “wickable mesh”. By “wickable mesh” is herein meant a mesh having a wicking time of less than 30 minutes (1800 seconds (sec)), preferably less than 20 minutes (1200 sec), and more preferably less than 15 minutes (900 sec), as determined using the method of measuring the wicking time described herein in the Examples section. A wickable mesh may have a wicking time of from 1 to 1800 sec, from 1 to 1200 sec, from 1 to 900 sec, from 1 to 700 sec, from 1 to 600 sec, from 1 to 550 sec, from 1 to 500 sec, from 1 to 400 sec, from 1 to 350 sec, from 1 to 300 sec, from 1 to 250 sec, from 1 to 200 sec, from 25 to 1800 sec, from 25 to 1200 sec, from 25 to 900 sec, from 25 to 700 sec, from 25 to 600 sec, from 25 to 550 sec, from 25 to 500 sec, from 25 to 400 sec, from 25 to 350 sec, from 25 to 300 sec, from 25 to 250 sec, from 25 to 200 sec, from 50 to 1800 sec, from 50 to 1200 sec, from 50 to 900 sec, from 50 to 700 sec, from 50 to 600 sec, from 50 to 550 sec, from 50 to 500 sec, from 50 to 400 sec, from 50 to 350 sec, from 50 to 300 sec, from 50 to 250 sec, from 50 to 200 sec, and any values within the foregoing ranges or any ranges created thereby.

The evaporative member is disposed in fluid communication with the transport member, preferably to the second end portion of the transport member. The transport member delivers the volatile composition from the reservoir 4 to the evaporative surface 3. The evaporative surface 3 may take different forms. Preferably the evaporative surface 3 presents a cylindrical configuration, this configuration has been found advantageous from a rate of delivery viewpoint. The dispenser can comprise a carriage to help retain the shape of the evaporative member.

The evaporative member comprises a woven mesh, preferably a woven wire mesh. The mesh comprises warps and wefts. Warps and wefts are the two basic components used in weaving to turn wires into a mesh. The lengthwise or longitudinal warps are held stationary in tension on a frame or loom while the transverse wefts (sometimes woof) are drawn through and inserted over and under the warp.

Mesh Count is the number of openings or apertures which can be counted per every linear inch of wire mesh. This is typically designated for both directions of the mesh. Therefore, a wire mesh which has ten openings per inch as measured across both its width and length would be designated a 10×10 mesh or Number 10 mesh.

Preferred meshes for use herein are off-count mesh. By “off-count mesh” is herein meant a wire mesh which has a different number of openings per inch in one direction than the other. Thus, a mesh with ten opening per inch measured in the length direction and seventeen openings per inch measured across its width would be designated a 10×17 mesh.

Preferred meshes for use herein have a number of opening per inch measured in the length direction of from 40-500, preferably from 50 to 400 and a number of openings per inch measured across the width direction of from 500-3000, preferably from 800-2500. The mesh may have a number of opening per inch measured in the length direction of from 40 to 500, from 40 to 400, from 40 to 300, from 40 to 200, from 40 to 100, from 40 to 50, from 50 to 500, from 50 to 400, from 50 to 300, from 50 to 200, from 50 to 100, from 100 to 500, from 100 to 400, from 100 to 300, from 100 to 200, from 200 to 500, from 200 to 400, from 200 to 300, from 300 to 500, from 300 to 400, from 400 to 500, and any values within the foregoing ranges or any ranges created thereby. The mesh may have a number of opening per inch measured in the width direction of from 500 to 3000, from 500 to 2500, from 500 to 2000, from 500 to 1500, from 500 to 1000, from 500 to 800, from 800 to 3000, from 800 to 2500, from 800 to 2000, from 800 to 1500, from 800 to 1000, from 1000 to 3000, from 1000 to 2500, from 1000 to 2000, from 1000 to 1500, from 1500 to 3000, from 1500 to 2500, from 1500 to 2000, from 2000 to 3000, from 2000 to 2500, from 2500 to 3000, and any values within the foregoing ranges or any ranges created thereby.

There are different types of woven meshes, including plain weave, twilled weave, dutch weave, etc.

In a plain weave each warp wire crosses alternately above and below every weft wire and vice versa. Warp and weft wires are normally of the same diameter. Plain weave meshes are normally described using its mesh count and wire thickness. The wire mesh count is the number of wires per square inch. And the wire thickness is in mm. For example a 10 #0.56 (the # stands for mesh), means 10 horizontal wires per inch and 10 vertical wires per inch. This mesh count gives 100 apertures (holes) per square inch.

As the woven mesh count goes up the wire thickness tends to decrease keeping the open area between 40 and 60 percent.

Twill Weave—Throughout the mesh, weft wires alternate above and below successive pairs of warp wires.

Dutch Weave—The dutch or ‘Hollander’ weave describes woven wire mesh in which the warp and weft wire diameters are different, as well as the number of meshes in the warp and weft directions. In the weaving process, the wires are driven much closer together, which results in a more densely compacted material. Most dutch weave wire mesh specifications are described in terms of mesh count per inch and aperture size in microns.

Twill Dutch Weave—In contrast to Plain Dutch Weave, there is a double layer of weft wires that are woven in a twill pattern. Twill Dutch Weave is “light tight,” having a very smooth surface, is strong, but has a restricted flow rate.

Reverse Dutch weave: reverse manner to Plain Dutch Weave. The weft wires are of greater diameter than the warp wires, and consequently the warp mesh count is greater than the weft mesh count.

During the course of this work, it has been found that plain weave meshes do not work. Without to be bound by theory, it is believed that the opening of the apertures of plain meshes are not suitable to promote wicking, in order to promote wicking the liquid path needs to be tortuous and not fully open as in the case of plain meshes. Twilled and Dutch meshes provide very good wicking.

One of the advantages of the evaporative member of the present disclosure is that the mesh does not act as a membrane, i.e., the mesh is not a selective barrier that allows some components to pass through but stops others. The mesh of the present disclosure allows the evaporation of the entire volatile liquid composition.

Preferably, the meshes of the present disclosure are not see-through with the naked eye.

The cross section of the warp and wefts can be circular, elliptical, square, rectangular, triangular, etc. Preferably the cross section of both the warp and wefts is cylindrical.

The mesh of the present disclosure can be made of any suitable material. It can be metallic or ceramic but preferably the mesh is metallic, more preferably made from stainless steel.

The transport member and the evaporative member of the present disclosure are capable of wicking a greater variety of perfume materials, leaving behind fewer, if any, perfume materials than traditional membranes. Traditional membranes that are selective, such as traditional polyethylene films, may inhibit high molecular weight volatile materials and materials with low solubility in polyethylene from diffusing through. This may limit perfume formulations, for example in the field of air fresheners where it is typically desired to use formulations having a wide variety of volatile materials having different volatilities. For example, some membranes made of traditional polyethylene films may preclude the diffusion of alcohols, such as linalool and dihydromyrcenol which are widely used in perfume applications.

While not wishing to be bound by theory, the physical characteristics of a membrane may affect the evaporation rate of volatile materials through the membrane. Such characteristics may include materials used, use of fillers, pore size, thickness, and evaporative surface area.

The mesh of the present disclosure may have a geometric pore size of about 5 to about 250 microns, preferably from 10 to 100 microns, more preferably from 10 to 50 microns. Mesh aperture [μm] or geometric pore size [μm] is defined as the diameter of the largest spherical particle that can pass through a mesh. This is calculated on the basis of the weave type, wire diameters and spacing parameters. The calculation equations that form the basis for this were developed and experimentally validated at the Institute of Mechanical Process Engineering at the University of Stuttgart within the scope of AVIF projects A 224 and A 251. If this calculation method does not apply for some meshes, the pore size is determined physically through the glass bead test.

The mesh of the present disclosure may have a porosity of from 10% to 60%, preferably from about 18 to about 58%. The mesh of the present disclosure may have a porosity of from 10% to 60%, from 10% to 58%, from 10% to 55%, from 10% to 50%, from 10% to 45%, from 10% to 40%, from 10% to 35%, from 10% to 30%, from 10% to 25%, from 10% to 20%, from 10% to 18%, from 10% to 15%, from 15% to 60%, from 15% to 58%, from 15% to 55%, from 15% to 50%, from 15% to 45%, from 15% to 40%, from 15% to 35%, from 15% to 30%, from 15% to 25%, from 15% to 20%, from 15% to 18%, from 18% to 60%, from 18% to 58%, from 18% to 55%, from 18% to 50%, from 18% to 45%, from 18% to 40%, from 18% to 35%, from 18% to 30%, from 18% to 25%, from 18% to 20%, from 20% to 60%, from 20% to 58%, from 20% to 55%, from 20% to 50%, from 20% to 45%, from 20% to 40%, from 20% to 35%, from 20% to 30%, from 20% to 25%, from 25% to 60%, from 25% to 58%, from 25% to 55%, from 25% to 50%, from 25% to 45%, from 25% to 40%, from 25% to 35%, from 25% to 30%, from 30% to 60%, from 30% to 58%, from 30% to 55%, from 30% to 50%, from 30% to 45%, from 30% to 40%, from 30% to 35%, from 35% to 60%, from 35% to 58%, from 35% to 55%, from 35% to 50%, from 35% to 45%, from 35% to 40%, from 40% to 60%, from 40% to 58%, from 40% to 55%, from 40% to 50%, from 40% to 45%, from 45% to 60%, from 45% to 58%, from 45% to 55%, from 45% to 50%, from 50% to 60%, from 50% to 58%, from 50% to 55%, from 55% to 60%, from 55% to 58%, from 58% to 60%, and any values within the foregoing ranges or any ranges created thereby. Porosity is defined as the ratio of empty space to total volume (empty volume compared to total volume and material) of a mesh. A high porosity generally also means high permeability. The mesh of the present disclosure may have a pore thickness of from about 47 to about 1200 microns (μm), preferably from about 80 to about 260 microns. The mesh of the present disclosure may have a pore thickness of from 47 to 1200 μm, from 47 to 1000 μm, from 47 to 800 μm, from 47 to 600 μm, from 47 to 400 μm, from 47 to 260 μm, from 47 to 200 μm, from 47 to 80 μm, from 80 to 1200 μm, from 80 to 1000 μm, from 80 to 800 μm, from 80 to 600 μm, from 80 to 400 μm, from 80 to 260 μm, from 80 to 200 μm, from 200 to 1200 μm, from 200 to 1000 μm, from 200 to 800 μm, from 200 to 600 μm, from 200 to 400 μm, from 200 to 260 μm, from 260 to 1200 μm, from 260 to 1000 μm, from 260 to 800 μm, from 260 to 600 μm, from 260 to 400 μm, from 400 to 1200 μm, from 400 to 1000 μm, from 400 to 800 μm, from 400 to 600 μm, from 600 to 1200 μm, from 600 to 1000 μm, from 600 to 800 μm, from 800 to 1200 μm, from 800 to 1000 μm, from 1000 to 1200 μm, and any values within the foregoing ranges or any ranges created thereby.

Preferably, each warp wire crosses alternately above and below every weft wire and vice versa. Warp and weft wires are normally of the same diameter.

Especially suitable for use herein are plain Dutch weaves. The mesh is manufactured as plain weave (1/1) and with a fineness of 45 μm to 300 μm absolute opening. The warp wires are interwoven with wider spaces than the weft wires. GKD Gebr. Kufferath AG, stocks plain dutch weave meshes from 45 μm to 300 μm made of stainless steels 304, 316 and 904 L. In contrast to square meshes, plain dutch weave is characterized by its significantly higher strength. Plain dutch weave meshes provide excellent wicking and liquid releasing properties.

Especially suitable for use herein are Dutch twilled weaves. Dutch twilled weave is a particularly robust mesh in relation to its fineness. These properties are realized through the high material density of this lightproof metallic mesh. The density is provided by a twilled weave (2/2) with a small number of thick warp wires and significantly more weft wires. GKD Gebr. Kufferath AG produces twilled Dutch weaves with a fineness of 5 μm to 250 μm absolute opening. Twilled Dutch weave is produced on the basis of the common materials in accordance with DIN 304 L/316 L

### Volatile Composition

In certain examples, the liquid volatile composition can comprise a single chemical and/or a single material that is capable of entering the vapor phase or, more commonly, the liquid volatile composition can comprise a mixture of chemicals and/or materials that are capable of entering the vapor phase. In various examples, the liquid volatile composition can comprise substances that can function as air fresheners, deodorants, odor neutralizing materials, odor blocking materials, odor masking materials, aromatherapy materials, aromachology materials, essential oils, insecticides, pesticides, pheromones, medicinals, flavors and/or combinations thereof. In other various examples, the liquid volatile composition can comprise other materials that can act in their vapor phase to modify, enhance, and/or treat an atmosphere or an environment. The dispenser can be configured for use in any environment, such as a domestic environment, for example, and can be configured to dispense any suitable solutions, chemical, materials, and/or compositions.

Suitable liquid volatile compositions are described in US 2010/0308130A1 and US 2010/0308126A1. Especially suitable liquid volatile compositions are described in U.S. Pat. No. 10,322,198, column 3, line 45 to column 12, line 28.

The volatile liquid composition may comprise from greater than 10 wt. %, alternatively greater than 20 wt. %, alternatively greater than 30 wt. %, alternatively greater than 40 wt. %, alternatively greater than 50 wt. %, alternatively greater than 60 wt. %, alternatively greater than 70 wt. %, alternatively greater than 85 wt. %, of perfume raw materials, based on the total weight of the freshening volatile liquid composition.

The dispenser can comprise more than one reservoir, each hosting a different, slightly different, or the same volatile liquid composition. Each volatile composition can comprise a different, slightly different, or the same vapor pressure range, for example. This feature can be useful when a user wants to dispense a first dose amount of a first volatile composition and a second dose amount of a second volatile composition, for example. In an instance in which more than one volatile compound is within one reservoir, the volatile composition with the higher vapor pressure range may transform from a liquid phase into a vapor phase prior to the volatile composition with the lower vapor pressure range transforming into a vapor phase. In this circumstance, the volatile composition with the higher vapor pressure range would likely be dispensed first, while the volatile composition with the lower vapor pressure range would likely be dispensed second. In various examples, where different volatile compositions with different vapor pressure ranges are in separate reservoirs, the different volatile compositions can be dispensed from their respective reservoirs simultaneously, for example. As a result, various volatile compositions can be dispensed from the dispenser to create a mixture of scents, for example, if the volatile liquid composition is a fragrance.

### Diffusion Assistance Means

The dispenser optionally comprises diffusion assistance means. The diffusion assistance means can be selected from the group consisting of heating elements, piezoelectric elements, fans and air pumps.

A fan assembly can comprise any suitable fan or components configured to produce and/or intermittently move a volume of air into the fan inlet and over the evaporative member.

In one example, the fan assembly can be housed in a fan housing. In various examples, the fan assembly can be positioned adjacent to the evaporative member or within 12 inches of the evaporative member, more preferably within 8 inches, more preferably within 6 inches, or more preferably within 3 inches of the evaporative member. The fan assembly may comprise a rotatable hub, and at least two fan blades extending from the rotatable hub or otherwise attached to or formed with the rotatable hub, and a motor.

The diameter of the rotatable hub may be about 8 mm to about 20 mm. The drive shaft can be operably engaged with the rotatable hub such that rotation of the drive shaft by the motor rotates the rotatable hub and thereby rotates the at least two fan blades.

The motor can provide continuous or intermittent movement of the fan blades to provide a volume of air over the evaporative member. In various examples, the fan assembly may produce air speeds in the range of about up to 5 cubic feet per minute (CFM). In one example, the fan can be a DAH20060S-W01 from Zhuzhou Yijie Electronic Technology Co., Ltd, the drive shaft rotates at about 7000 revolutions per minute when 3.7 VDC and 55% duty cycle is supplied to the motor from the power source and rotates the driveshaft at about 13000 revolutions per minute when 3.7 VDC and 100% duty cycle is supplied to the motor from the power source. In various examples, the flow velocity of the air generated by the motor can be in the range of about 0.7 to about 1.0 m/sec at about 55% duty cycle to about 1.5 to about 2.2 m/sec at 75% duty cycle, depending upon the cross sectional area of the inlet orifice and the outlet orifice. By supplying various duty cycle to the motor, the rotational speed of the drive shaft and the resultant flow rate of the volume of air can be varied. Additionally, the controller can supply the motor with voltage using any suitable technique known to those of skill in the art. In various examples, a pulse width modulation technique can be used to provide voltage to the motor over a specified range, such as about 4.5 VDC to about 5.5 VDC, for example. Additional circuitry or components, such as an analog-to-digital converter, can be used to compensate for various factors, such as the power source voltage and the ambient temperature, for example. In order to isolate or limit vibration due to the rotation of the drive shaft and/or the rotatable hub, vibration suppression devices or techniques can be used, such as silicon or thermoplastic elastomeric fan supports, for example, and/or the use of a gasket at the interface of the delivery engine and the housing.

In one example, the fan assembly can comprise a centrifugal (i.e., radial) fan. Each fan blade can comprise an air forcing surface that is positioned in a direction parallel to, or substantially parallel to, an axis of rotation of the rotatable hub. In one example, an electrical current can be provided to the motor via electrically conductive leads or terminal to rotate the rotatable hub. Such rotation can cause a volume of air to be drawn into the fan inlet and forced in a radial direction relative to the drive shaft. In other examples, the volume of air can be drawn from the atmosphere outside of the dispenser through any suitable vent or passageway on the housing 1, for example. The rotation of the at least two fan blades can force the volume of air out of the fan housing through the fan outlet and over the evaporative member. In various examples, the at least two fan blades can be arcuate, straight, and/or can have curved, straight, and/or arcuate portions. Additionally, the at least two fan blades can have various cross-sectional shapes, such as an airfoil shape or a tapered shape, for example. As will be appreciated by those of skill in the art, after consideration of the present disclosure, a centrifugal fan can provide high efficiency with relatively small dimensions, and changes in pressure may have little influence on pressure head drops through the dispenser.

In another example, the fan assembly can be an axial fan. This axial fan can comprise a rotatable hub and at least three fan blades extending from the rotatable hub. This at least three fan blades are attached to or formed with the rotatable hub. In one example, the diameter of the rotatable hub can be about 8 mm to about 20 mm, for example, although other dimensions could be possible. The fan assembly can define a fan inlet. The drive shaft can be operably engaged with the rotatable hub such that rotation of the drive shaft by the motor rotates the rotatable hub and thereby rotates the at least three fan blades. In various examples, the fan assembly may produce air flows in the range of 0.1 CFM to 5 CFM, or 0.2 CFM to 2 CFM, or 0.4 CFM to 1.0 CFMf; although others air speeds could be possible.

Each blade can comprise an air forcing surface that is positioned in a direction perpendicular to, or substantially perpendicular to, an axis of rotation of the rotatable hub. In one example, an electrical current can be provided to the axial motor via electrically conductive leads or terminal to rotate the rotatable hub. Such rotation can cause a volume of air to be drawn into the fan housing through the fan inlet. With an axial fan configuration, the air flowing through the fan assembly can be drawn through the fan inlet and forced to move along the drive shaft direction. The rotation of the at least three fan blades can force the volume of air out of the fan housing through the fan outlet and over the evaporative member. In various examples, the at least three fan blades can be arcuate, straight, and/or can have curved, straight, and/or arcuate portions. Additionally, the at least three fan blades can have various cross-sectional shapes, such as an airfoil shape or a tapered shape, for example. As will be appreciated by those of skill in the art, after consideration of the present disclosure, an axial fan can provide high efficiency with relatively small dimensions, and changes in pressure may have little influence on pressure head drops through the delivery engine.

Suitable fans for the present disclosure include a 20×20×6 mm DAH20060S fan.

The fan assembly can be powered by a power source which may comprise an AC/DC outlet, a battery, or series of batteries, such as an AA battery, an AAA battery, a 9-volt battery, rechargeable battery, such as lithium/lithium ion batteries, and/or other suitable battery. In one example, a solar power source, such as a solar cell, for example, can be used to power the device. In various examples, the solar cell (i.e., a photovoltaic cell) can be positioned on an outer portion of the dispenser or in communication with the device, such that the solar cell can receive light that can be transformed into energy to power the dispenser. Those of skill in the art, upon review of the present disclosure, will recognize that any other suitable method or device can be used to provide power to the dispenser.

In various examples, the control technique or approach for the fan can be at least based on characteristics of the volatile composition. Volatile compositions with lower vapor pressures will likely evaporate slower than volatile compositions with higher vapor pressures. In various examples, the fan assembly may not be activated until the evaporative member has reached full saturation or near full saturation of the volatile composition. In one example, the deactivation time period of the fan can be related to the time period necessary for the volatile composition to evaporate and saturate, or at least partially saturate, the space with the vapor phase volatile composition. In one example, the activation time period of the fan assembly can be related to the time period necessary to expel substantially all of the vapor phase volatile composition from the reservoir into the atmosphere. Once the vapor has been expelled from the reservoir, the fan assembly can be placed in an inactive state to again allow a portion of the volatile composition to enter the vapor phase.

By activating the fan assembly for a period of time equal to, or approximately equal to, the amount to time necessary to expel at least most of the vapor phase volatile composition, the lifetime of the power source can be optimized. Through control of the fan assembly, maximum vapor phase volatile composition release can be achieved with a minimum amount of fan assembly running time. In various examples, the sequencing or pattern of activator actuation, or the flow rate of the volume of air produced by the fan assembly, can be adjusted to allow full or near full saturation of the volatile composition within the space for maximizing the vapor phase volatile composition release. In one example, the fan assembly can be activated for about 1 to about 10 seconds and then deactivated for about 1 to about 10 seconds, for example.

In various examples, the duration of activation of the fan assembly or the flow rate of the volume of air provided by the fan assembly can be increased to provide a higher intensity of volatile composition expulsion from the dispenser. The fan assembly can operate continuously or have intermittent operation. The fan assembly may toggle on and off for a duty cycle of about 5% to about 50%, or from about 8% to about 20%. By providing a period of time between consecutive activations of the fan assembly, a user is more likely to notice a scent of the volatile composition again and avoid habituation.

In various examples, the evaporation rate of a liquid volatile composition from the dispenser can be about 1 mg/hr to about 100 mg/hr, or about 3 mg/hr to about 60 mg/hr, or about 5 mg/hr to about 40 mg/hr, or about 10 mg/hr to about 40 mg/hr, or about 15 mg/hr to about 40 mg/hr, and any values within the foregoing ranges or any ranges created thereby.

It is contemplated that other diffusion assistance means can be utilized to achieve the evaporation rate of a volatile composition from a dispenser of the present disclosure. Such diffusion assistance means may include an agitation member or agitator, both powered agitator and manual agitator, to assist with agitating the liquid volatile composition in the reservoir. The diffusion assistance means may also include a heating element to heat the liquid volatile composition, a chemical constituent to speed evaporation or release rates, use of a chemically heated mesh to provide increased evaporation via exothermic reaction, or synergistic combinations thereof.

In various examples, a controller may be positioned in electrical communication with the fan, such that the controller can instruct the fan when to activate and which speed to rotate to force the volume of air over the evaporative member. In one example, the controller can be any suitable type of controller, such as a microcontroller, for example. In one example, the controller can be a Texas Instruments MSP430F2132 controller. In various examples, the controller can comprise one or more user input buttons or switches configured to provide an input signal to the controller when depressed by a user, such that the controller can send corresponding output signals to the fan assembly and/or the user feedback module, for example. In one example, the various user input buttons or switches can comprise a power on/off switch configured to power on or power off the dispenser and at least one volatile composition dose amount button configured to allow the user to adjust the amount of volatile composition dispensed by the dispenser. As will be appreciated, the input buttons or switches can be any combination of buttons and/or switches, such as push buttons, sliders, dials, knobs, for example.

In some examples, a communication network may be implemented to gather information about the dispenser for the user. For example, the dispenser may be configured with a central device controller to form ad hoc, wireless mesh networks and control multiple communication modules. For example, the central device controller may be in communication with sensors to sense the amount of a volatile composition that has evaporated into a room having the device.

In various examples, the amount of the liquid volatile composition dispensed over a predetermined time interval can be controlled by adjusting the rate at which the fan assembly is activated by the controller (i.e., by adjusting the time period the fan is active and the time period the fan is inactive), by adjusting the speed at which the air is moved when the fan assembly is active (i.e., by adjusting the rotational speed by adjusting the voltage to the motor), and/or by a combination of both techniques. In one example, the dispenser can have a “boost” button for delivering a dose of the volatile composition to the atmosphere on demand. For example, if the boost button is depressed or otherwise activated, the fan assembly can be activated for a specified time period, such as 30 to 60 seconds or at a specified rotational speed, for example.

The dispenser can be configured to deliver a volatile liquid composition into the air with increased noticeability over the life of the volatile composition contained within a reservoir. It has been found that varying the energy applied to the volatile liquid composition over a total emission cycle can affect the consumer noticeability of the volatile composition over time. In particular, an initial energy boost period applied to the volatile composition, followed by a decrease in energy for an extended emission period, with successive energy boosts and variation in energy over a period results in improved noticeability of the volatile liquid composition by the user.

The diffusion assistance means, such as a heater or fan, may be programmed to operate in various operational conditions. As will be discussed in more detail below, the evaporative assistance elements may be configured to have various discrete emission periods, gaps in emission of any evaporative assistance elements, varying energy profiles over time, randomized energy profiles, simultaneous emission periods, and combinations thereof. Each of these methods of operation, either alone or in combination, may promote user noticeability of the volatile composition and/or reduce the likelihood of short-term or long-term habituation of the volatile composition.

The term “discrete emission period”, as used herein, refers to the individual time period that a given volatile composition is emitted in an emission sequence. This may correspond generally to the period of time that a diffusion assistance means is turned ON for a given fill of volatile composition, although there may be a slight lag between the operation of a diffusion assistance means and the emission of a volatile composition. The term “extended emission periods”, as used herein, includes a plurality of successive discrete emission periods that may be separated by gaps in operation where the evaporative assistance element is OFF.

The “total emission program” refers to the entire sequence, including all discrete emission periods and OFF times for gaps in emission that make up the energy boosts and extended emission periods, from beginning to end of life of a “filled” volume of volatile composition in a reservoir. “Fill” or “filled” as used herein refers to an amount of volatile composition that is intended to occupy the whole of or substantially the whole of the available volume in the reservoir, which excludes any volume occupied by any other elements of the volatile composition dispenser that may be disposed in the reservoir, such as the delivery engine. The reservoir will typically be occupied or filled to least 80%, 85%, 90%, or 95% volatile composition, of the total available volume of the reservoir. The total emission program is then designed to evaporate all or substantially all of the volatile composition in the reservoir.

The total emission program may be continuous. The term “continuous”, as used in reference to the emission program, means that there is a planned emission sequence over an entire period, once the program is initiated. This emission program can include periods, as noted above, where there are gaps in emission. This will still be considered to be a continuous emission program, although there will not necessarily be continuous emission of volatile compositions. It should be understood, however, that it is possible for the emission program to be interruptible by the user (e.g., turned off), if desired. Thus, the method can provide a user interface, and the user interface can provide a user with the ability to interrupt emission program. The emission program may be designed to run continuously, or substantially continuously until at least one of the volatile compositions is substantially depleted from the reservoir. It may be desirable for the emission program to run continuously until all of the volatile compositions are substantially depleted, and for this to occur at approximately the same time.

If the total emission program is disrupted, the dispenser may be configured with memory to record the last emission sequence that was initiated in the event that the volatile composition dispenser is disconnected from the power source. Once operation of the volatile composition dispenser is resumed, the memory of the last recorded sequence is recalled to return the total emission program to the correct emission sequence. The total emission program may only be restarted at the beginning of the program when a new or refilled reservoir/cartridge is installed into the housing.

The total emission program can be of any suitable length, including but not limited to 10 days, preferably 15 days, preferably 20 days, preferably 25 days, preferably 30 days, more preferably 45 days, more preferably 60 days, more preferably 90 days, more preferably 130 days, more preferably 150 days, or shorter or longer periods, or any period between 30 to 150 days, and any values within the foregoing ranges or any ranges created thereby.

The discrete emission period for each diffusion assistance means in a volatile composition dispenser may be in the range of 2 minutes to 48 hours, alternatively 5 minutes to 48 hours, alternatively 10 minutes to 48 hours, alternatively 15 minutes to 48 hours, alternatively 20 minutes to 24 hours, alternatively 30 minutes to 8 hours, alternatively 45 minutes to 4 hours, and any values within the foregoing ranges or any ranges created thereby. The higher the energy supplied by the diffusion assistance means, such as a higher temperature supplied by a heater, the shorter the discrete emission period that may be needed to provide a noticeable amount of volatile composition into the air.

During the discrete emission period for a particular diffusion assistance means, the diffusion assistance means will be continuously ON. In a volatile composition dispenser comprising more than one diffusion assistance means, the diffusion assistance means may have alternating discrete emission periods. In an alternating system, one diffusion assistance may be turned ON while the other diffusion assistance means may be turned OFF. Or, one or more diffusion assistance means may be turned ON at a given time. The operation of two or more diffusion assistance means may overlap for a period of time. The greater the discrete emission period for each diffusion assistance means, the potential for higher concentrations of volatile composition in the surrounding space in order to increase user noticeability. There may also be time periods when all diffusion assistance means are turned OFF. Each diffusion assistance means may be configured to have the same discrete emission period, or some or all of the diffusion assistance means may be configured to have different discrete emission periods.

Evaporation rates of the volatile composition from the evaporative member may be about 1 mg/hr to about 100 mg/hr, or about 3 mg/hr to about 60 mg/hr, or about 5 mg/hr to about 40 mg/hr, or about 10 mg/hr to about 40 mg/hr, about 15 mg/hr to about 40 mg/hr, about 20 mg/hr to about 40 mg/hr, and any values within the foregoing ranges or any ranges created thereby.

Near the end of the total emission program, the volatile composition dispenser may operate at or near the maximum power output, such as maximum temperature or fan speed, until unplugged and a new cartridge or reservoir is installed.

The total emission program may be configured to turn OFF an evaporative assistance element when the volatile composition is depleted from the reservoir. For example, the evaporative assistance element may turn OFF after a predetermined time period for a given intensity setting. By turning OFF the diffusion assistance means, energy is not applied by the diffusion assistance means until the reservoir is refilled or replaced with a new fill of volatile composition.

In various examples, the controller can also be in electrical communication with a temperature sensor configured to sense the temperature of the atmosphere. In various examples, the temperature sensor can send a signal to the controller indicative of the temperature of the space, such that the controller can provide an output signal to the fan assembly or other various components of the dispenser, indicative of a volatile composition dosing amount for a particular temperature and/or temperature range. For example, higher temperature ranges may require greater dose amounts than lower temperature ranges to achieve the desired result. As a result, the dispenser can be power efficient such that it can maximize the life of the power source. The dispenser can be activated for 1-30 seconds, for example, and then be inactive for 10-200 seconds, for example. In other various examples, the dispenser can be set by a user to provide a desired intermittent dosing amount.

In various examples, the dispenser can comprise a sensor, such as a visible indicator, a light source, and/or an audible alert, configured to provide feedback to the user regarding the status of the dispenser or the room occupancy. In one example, the sensor can be used to alert the user of a property of the dispenser. In such examples, the feedback can be visual and/or audible and can indicate to the user, among other things, whether the dispenser is powered on, what volatile composition dosing amount is being dispensed, the power level of the power source, the amount, type, or level of the volatile composition within the delivery engine, and/or any other suitable feedback helpful or beneficial to the user. In various examples, the sensor can comprise one or more one indicators, such as a plurality of light sources, for example, electrically coupled to the controller and/or to the power source, and a translucent portion in the housing, such that the one or more indicators can be viewed by the user though the housing. In one example, the one or more indicators can be oriented in any suitable fashion such that various lights of the one or more indicators can emit visible light through the translucent portion of the housing, depending on what type of feedback is being provided to the user. In one example, the translucent portion of the housing can comprise any suitable shape and the one or more indicators can be arranged in a similar shape so that as one indicator, such as a light two or more light sources are powered or unpowered, the user is provided with at least a second feedback and so forth. In one example, at least one button is at least partially translucent allowing for one or more indicators to be viewable through the button.

With some liquid volatile compositions (for instance those comprising fragrances) it may be helpful to adjust the fan speed, frequency of run time, or on/off time to compensate for the changing volatile composition formulation as high vapor pressure volatile composition raw materials will evaporate more quickly than low vapor pressure raw materials. In this case it may optionally be desirable to have the controller operate the fan more frequently as the volatile composition is evaporated over a period of many days. For instance, in one non-limiting example, the fan could run at 10% duty cycle for the first 10 days of usage but then slowly increase up to about 30% to about 40% duty cycle from days 11 up to 60 days. In this way, the fan frequency or duration can be increased to compensate for potentially a decline in fragrance intensity. By adjusting for the age, it is possible to deliver a more consistent scent intensity even as the fragrance amount and mixture of high to low vapor pressure components is changing with time. One non-limiting example of a means of keeping track of run time of the delivery engine is to monitor the voltage of the battery associated with the delivery engine. For instance, a fully charged Li-ion battery may be 3.60 Volts to about 3.7 Volts while a Li-ion battery that was used for thirty days might have a voltage of lower than 3.45 Volts. By monitoring the voltage of the battery, the controller can recognize the life of the delivery engine and can adjust operating conditions to deliver a consistent scent experience over the life of the delivery engine.

Another non-limiting example of a means to monitor time, is to start a timer when the delivery engine is inserted and to keep track of hours/minutes that the fan has operated. As mentioned above, the fan time could be adjusted as the product ages to deliver a more consistent scent experience.

In the instance where the battery voltage or run time is viewed as the indicator of the full life of the delivery engine, the controller could be programmed to provide a signal to the user such as turning on a red light or provide a flashing light to indicate that the delivery engine is empty and/or needs to be replaced.

Surprisingly, the dispenser of the present disclosure requires low power and/or low current consumption. This makes it suitable to be powered by batteries. It makes it very attractive because it does not require to be plugged in and therefore it is very versatile in terms of where to place it. Preferably, the dispenser of the present disclosure evaporates from 5 to 40 mg/hr, or from 10 to 40 mg/hr with a power consumption less than 200 mA, or less than 20 mA or less than 10 mA and/or with a power source having a max voltage of less than 24V or less than 10V or less than 6V.

### Methods

The present disclosure also includes a method of evaporating a liquid volatile composition into a space.

The method may comprise the step of providing a dispenser comprising:
i. a housing
ii. a reservoir holding the liquid composition;
iii. a transport member for the liquid composition;
iv. an evaporative member in fluid communication, preferably in fluid contact with the transport member wherein the evaporative member draws the liquid composition from the transport member and wet the evaporative member with the liquid composition; and
v. the liquid composition evaporates from the evaporative member.

Preferably, the method of the present disclosure uses the dispenser of the present disclosure.

### Preferred Combinations

A. A dispenser for a volatile liquid composition, the dispenser comprising:
   a) a housing;
   b) a reservoir holding the liquid composition;
   c) a transport member for the liquid composition; and
   d) an evaporative member in fluid communication with the transport member wherein the evaporative member comprises a wickable mesh.
B. The dispenser according to paragraph A, wherein the mesh is a woven mesh.
C. The dispenser according to paragraph A or B, wherein the mesh is a wire mesh comprising warp and weft wires.
D. The dispenser according to paragraph C, wherein the warp and wefts have a substantially circular cross section.
E. The dispenser according to any of paragraphs A to D, wherein the mesh is an off-count mesh.
F. The dispenser according to any of paragraphs A to E, wherein the mesh is selected from plain Dutch weave, reverse plain Dutch weave, Dutch twilled weave and reverse Dutch twilled weave.
G. The dispenser according to any of paragraphs A to F, wherein the mesh is not see-through.
H. The dispenser according to any of paragraphs A to G, wherein the mesh has a geometric pore size of from about 5 to about 250 microns, preferably from 10 to 100 microns.
I. The dispenser according to any of paragraphs A to H, wherein the mesh has a porosity of from about 10% to about 60%.
J. The dispenser according to any of paragraphs A to I, wherein the mesh is metallic or ceramic.
K. The dispenser according to any of paragraphs A to J, wherein the mesh is made of stainless steel.
L. The dispenser according to any of paragraphs A to K, wherein the evaporative member has a cylindrical configuration.
M. The dispenser according to any of paragraphs A to L, wherein the transport member comprises a wick.
N. The dispenser according to any of paragraphs A to M, wherein the transport member comprises a wick and the wick is made of a material selected from the group consisting of fibers, plastic, glass, metal, rubber, silicone, and combinations thereof.
O. The dispenser according to any of paragraphs A to N, further comprising diffusion assistance means.
P. The dispenser according to any of paragraphs A to O, further comprising diffusion assistance means wherein the diffusion assistance means are selected from the group consisting of heating elements, piezoelectric elements, fans and air pumps.
Q. The dispenser according to any of paragraphs A to P, further comprising diffusion assistance means wherein the diffusion assistance means generates airflow directed at the evaporative member.
R. The dispenser according to any of paragraphs A to Q, further comprising diffusion assistance means wherein the diffusion assistance means comprises a fan.
S. The dispenser according to any of paragraphs A to R, further comprising diffusion assistance means wherein the diffusion assistance means further comprises an energy source for heating.
T. The dispenser according to any of paragraphs A to S, wherein the housing comprises at least one inlet opening for attracting air and at least one outlet opening for directing the airflow generated by the fan to the environment.
U. The dispenser according to any of paragraphs A to T, wherein the dispenser is battery operated.
V. A dispenser for a volatile liquid composition, the dispenser comprising:
   a housing
   a reservoir holding the liquid composition;
   a transport member for the liquid composition;
   an evaporative member in communication with the transport member wherein from about 5 to about 40 mg/hr of volatile composition is evaporated consuming less 20 mA and/or using a power source with a max voltage less than 24V and wherein the evaporative member preferably comprises a wickable mesh, more preferably the mesh, is a mesh as that of dispensers according to paragraphs B to K.
W. A method for dispensing a volatile liquid composition using a dispenser comprising:
   a housing
   a reservoir holding the liquid composition;
   a transport member for the liquid composition;
   an evaporative member in communication with the transport member
   the method comprising the step of wetting the evaporative member with the liquid composition; and
   evaporating the composition from the evaporative member, wherein the evaporative member preferably comprises a wickable mesh, more preferably the mesh, is a mesh as that of dispensers according to paragraphs B to K.

### EXAMPLES

The wicking times of a perfume composition through different meshes were measured to evaluate the suitability of meshes as evaporative members to dispense a perfume composition.

### Method of Measuring the Wicking Time

Gloves should be worn when handling the mesh sheets and cut samples as oils from the skin could affect results. For each sheet of mesh sample to be tested, label one side as the ‘X Axis’ and the other side (90 degree turn) as the ‘Y-Axis’ side. Cut three samples to 7 cm×1 cm size from the X-Axis side and the Y-Axis side (i.e. there will be 3 samples with the 7 cm cut length from the X-Axis side of the mesh and 3 samples with the 7 cm cut length from the Y-Axis side of the mesh). From the bottom edge of the longer cut side (7 cm), mark a line up at 3 cm. In a 50 ml beaker, add 5 g of a composition perfume. The perfume composition used for the testing is non-aqueous and contains at least 10% dipropylene glycol methyl ether solvent and has a viscosity of approximately 4.8 cPs and a density of approximately 0.92 g/ml. A suitable example of a perfume composition for this testing is from the Febreze PLUG™ Linen & Sky refill. Suspend the mesh sample with the 7 cm cut side vertically positioned above the perfume composition, and confirm that the sample is hanging level. Lower the mesh sample so that the bottom edge should touch the top surface of the liquid perfume composition. The mesh sample should be lowered no more than 1-2 mm into the liquid. Start the timer as soon as the mesh is in contact with the perfume composition, and record the time for the perfume composition to wick upward to the 3 cm line. The testing is repeated with each of the 3 samples cut from the X-Axis side and each of the 3 samples cut from the Y-Axis side of the mesh. For each mesh material, the wicking time reported is the average wicking time across the 6 samples. Any mesh with at least one sample that does not wick to the 3 cm line after 30 minutes does not receive an average wicking time and is marked as not wicking out. All testing is conducted at room temperature 21° C.+/−2° C. A small amount of an oil-soluble dye can optionally be added to the perfume composition to make it easier to visualize the perfume composition wicking out across the mesh.

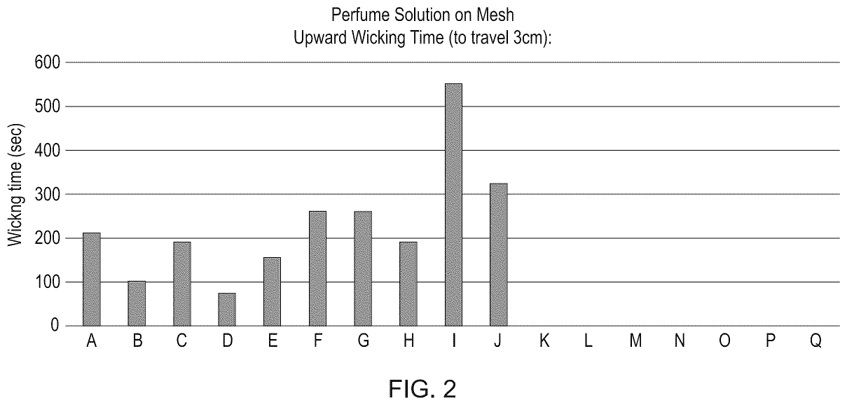

The wicking time (sec) of the mesh samples described below are shown in the graph in FIG. 2.

The manufacturer and descriptor of meshes A to Q are:
A: Taiwan: Twill Dutch Weave 165×1400 mesh
B: Taiwan: Twill Dutch Weave 200×600 mesh
C: G. Bopp: Twilled Dutch Weave 165×1400 mesh
D: McMaster: SS316 80×700 mesh
E: McMaster: SS316 165×1400 mesh
F: McMaster: SS316 325×2300 mesh
G: GKD: PZ-17 19 micron
H: GKD: OT-20 21 micron
I: GKD: OT-30 25 microns
J: GKD: PZ-40 (290/72)
K: McMaster: 304 SS 150×150 Mesh
L: McMaster: 304 SS 200×200
M: McMaster: 304 SS 250×250
N: McMaster: 316 SS 325×325
O: McMaster: 316 SS 400×400
P: McMaster: 6S500M0008 (CP91-68)
Q: McMaster: SS100M0045 (CP75)

Meshes A to J are wickable meshes, as shown by FIG. 2. Meshes K to Q are not, meaning that they failed to wick upwards, therefore not having a wicking time.

### Evaporation Rate Test Method

1) Mesh samples were cut into test swatches of 3 cm×3 cm
2) Mesh square was placed on top of a wick, ensuring complete contact with mesh/wick top.
3) A small plastic “Mesh-Holder” was placed on top of a wick to hold the mesh in place (the “Mesh Holder” is a plastic part that clamps to the top of a perfume refill bottle and slides overtop of the wick (without touching the wick) and has a plastic protrusion on the top which pushes the mesh firmly onto the surface of the mesh and secures it in place.
4) The test unit (Bottle containing the perfume composition+Wick+ “Mesh-Holder”+Mesh) was weighted and the weight and time recorded.
5) The sample was placed in a rectangular chamber with controllable airflow and an airflow of ˜ 1.7 m/s was applied to the area of the chamber where the test sample was located. The sample remained in the airflow chamber for 24 hours.
6) After ˜24 hours (Step 5, above) the test unit with mesh was removed and the final weight (g) after overnight run was recorded.
7) The average release rate was calculated by dividing the weight change from the initial time point to the final time point (24 hours)=((Weight Initial (mg)−Weight Final (mg))/(Elapsed time in hours))

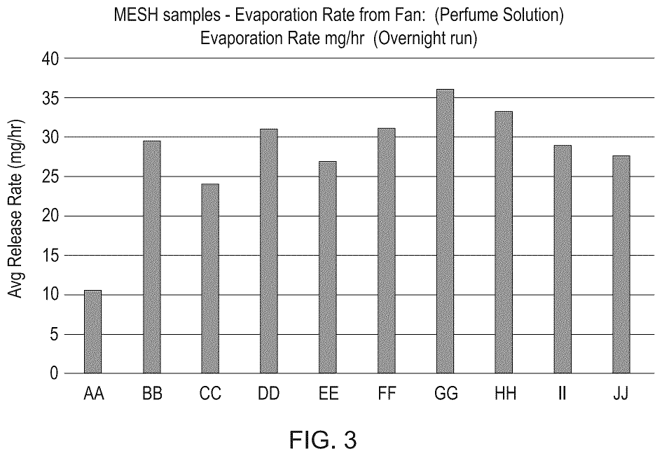

The average release rate (mg/hr) of the samples described below are shown in the graph in FIG. 3.
AA: Control, exposed wick with no mesh
BB: Taiwan: Twill Dutch Weave 165×1400 mesh
CC: Taiwan: Twill Dutch Weave 200×600 mesh
DD: G. Bopp: Twilled Dutch Weave 165×1400 mesh
EE: McMaster: SS316 325×2300 mesh
FF: EE: McMaster: SS316 165×1400 mesh
GG: K: McMaster SS316 80×700 mesh
HH: GKD: PZ-17 19 micron
II: GKD: OT-20 21 micron
JJ: GKD: OT-30 25 microns

FIG. 3 shows that wickable meshes present very good evaporation rate of perfume as compared to just a wick with no mesh (AA).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A dispenser for a volatile liquid composition, the dispenser comprising:
a housing;
a reservoir holding the liquid composition;
a transport member for the liquid composition; and
an evaporative member in fluid communication with the transport member wherein the evaporative member comprises a wickable mesh.

2. The dispenser according to claim 1, wherein the mesh is a woven mesh.

3. The dispenser according to claim 1, wherein the mesh is a wire mesh comprising warp and weft wires.

4. The dispenser according to claim 1, wherein the mesh is a wire mesh comprising warp and weft wires and the warp and wefts have a substantially circular cross section.

5. The dispenser according to claim 1, wherein the mesh is an off-count mesh.

6. The dispenser according to claim 1, wherein the mesh is selected from plain Dutch weave, reverse plain Dutch weave, Dutch twilled weave and reverse Dutch twilled weave.

7. The dispenser according to claim 1, wherein the mesh is not see-through.

8. The dispenser according to claim 1, wherein the mesh has a geometric pore size of from about 5 to about 250 microns.

9. The dispenser according to claim 1, wherein the mesh has a porosity of from about 10% to about 60%.

10. The dispenser according to claim 1, wherein the mesh is metallic or ceramic.

11. The dispenser according to claim 1, wherein the mesh is made of stainless steel.

12. The dispenser according to claim 1, wherein the evaporative member has a cylindrical configuration.

13. The dispenser according to claim 1, wherein the transport member comprises a wick.

14. The dispenser according to claim 1, wherein the transport member comprises a wick and the wick is made of a material selected from the group consisting of fibers, plastic, glass, metal, rubber, silicone, and combinations thereof.

15. The dispenser according to claim 1, further comprising diffusion assistance means selected from the group consisting of heating elements, piezoelectric elements, fans, and air pumps.

16. The dispenser according to claim 1, further comprising diffusion assistance means wherein the diffusion assistance means generates airflow directed at the evaporative member.

17. The dispenser according to claim 1, further comprising diffusion assistance means wherein the diffusion assistance means comprises a fan.

18. The dispenser according to claim 1, further comprising diffusion assistance means wherein the diffusion assistance means further comprises an energy source for heating.

19. The dispenser according to claim 1, wherein the housing comprises at least one inlet opening for attracting air and at least one outlet opening for directing the airflow generated by the fan to the environment.

20. The dispenser according to claim 1, wherein the dispenser is battery operated.
